(19) United States
(12) Patent Application Publication
(10) Pub. No.: US 2006/0263839 A1
(43) Pub. Date: Nov. 23, 2006

(54) COMBINED DRUG DELIVERY AND ANALYTE SENSOR APPARATUS

(75) Inventors: W. Kenneth Ward, Portland, OR (US); Mark Neinast, Lake Oswego, OR (US); Richard Sass, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: iSENSE CORPORATION, Portland, OR (US)

(21) Appl. No.: 11/382,674

(22) Filed: May 10, 2006

Related U.S. Application Data
(60) Provisional application No. 60/682,209, filed on May 17, 2005.
(60) Provisional application No. 60/735,310, filed on Nov. 10, 2005.

(51) Int. Cl. C12Q 1/54; A61B 5/04

(52) U.S. Cl. 435/14; 600/372

(57) ABSTRACT

Embodiments of the present invention provide methods and apparatuses for analyte sensing combined with drug delivery in an integrated system. In an embodiment, a device may be utilized to sense an analyte, and in response to a measurement obtained therefrom, introduce a controlled amount of a drug to a user as a corrective action.

### CROSS REFERENCE TO RELATED APPLICATIONS

The present application claims priority to U.S. Provisional Patent Application No. 60/682,209, filed May 17, 2005, entitled “Lactate Sensing Intravenous Catheter,” and U.S. Provisional Patent Application No. 60/735,310, filed Nov. 10, 2005, entitled “Combined Drug Delivery and Analyte Sensor Apparatus,” the entire disclosures of which are hereby incorporated by reference in their entirety.

### TECHNICAL FIELD

Embodiments of the present invention relate to medical devices, more specifically, to methods and apparatuses for providing analyte sensing combined with drug delivery.

### BACKGROUND

Sensing of analyte in situ is desirable to reduce the need for extraneous equipment or devices. Typically, in order to measure analyte in a body, a sample is drawn from the body and measured using an external device. Furthermore, if any corrective action is deemed appropriate, typically a second device is utilized to introduce a corrective drug into the body.

For example, for patients with diabetes who take insulin, the process of treating their condition is quite complex. They must keep track of the amount of carbohydrates and other nutrients that they ingest; they must monitor capillary blood glucose values by repeated lancing of fingers or other body sites; and they must take into consideration the amount of exercise in which they engage. They must take into consideration all these factors in order to compute the doses of insulin that they administer regularly. If the glucose concentration is not well controlled and is chronically elevated, they run a risk of developing long term complications such as disease of the eyes, kidneys, nerves, feet and heart. If their blood glucose concentration falls too low, they run a risk of, for example, experiencing seizures, coma and automobile accidents.

For all these reasons, a system that could deliver the correct amounts of insulin with little or no patient interaction would be helpful to a person with insulin-treated Type 1 or Type 2 diabetes. However, automated pancreas systems have been quite cumbersome to date. For example, in the late 1970's a large device known as the BIOSTATOR was developed and was able to measure glucose on a continuous or near-continuous basis by withdrawing and measuring venous blood glucose values. See Fogt E J, Dodd L M, Jenning E M, Clemens A H, Development and evaluation of a glucose analyzer for a glucose controlled insulin infusion system (BIOSTATOR), Clin. Chem., 1978 August;24(8):1366-72. In addition, the BIOSTATOR was able to administer insulin. Because of its size, the BIOSTATOR was relegated to a research tool and was never able to achieve widespread use among people with diabetes.

In more recent years, other attempts have been made to integrate a glucose sensor and an insulin infusion device. One such system was described by Hovorka and colleagues (Hovorka R, Chassin L J, Wilinska M E, et al., Closing the Loop, the Adicol Experience, Diabetes Technol. Ther., 2004 June;6(3):307-18). In this system, a temporarily-implanted needle-type glucose sensor (microdialysis-type) was combined with a hand held computer and a belt-worn insulin pump in order to close the loop. One limitation of a microdialysis-type sensor is that it is a complicated device that requires fluid delivery into the microdialysis catheter, and fluid removal from the microdialysis catheter.

Steil and colleagues have also described a complex closed loop system, in which an intravenous sensor or subcutaneous sensor is combined with a fully-implantable or an external insulin pump and a computer (Steil G M, Panteleon A E, and Rebrin K, Closed-loop insulin delivery—the path to physiological glucose control, Adv Drug Deliv Rev, 2004 Feb. 10;56(2):125-44). However, such a system requires two separate units: one for the insulin pump (and catheter) and one for the sensing apparatus (which may use a separate catheter for sensing).

In other environments, such as sensing of lactate, similar desirability for sensing of analyte in situ and delivery of drugs may arise. For example, it has been found that blood loss leading to reduced perfusion (circulation) is often not apparent, and thus has been termed occult hypoperfusion (OH). OH is quite common in trauma patients and it often leads to death. However, if, when elevated blood levels of lactic acid are first detected, a medical team intervenes quickly, then the source of OH can often be found and the life of the patient saved.

The reason that blood lactate rises when the blood volume is reduced is related to oxygen supply and demand. Normally, the lungs oxygenate blood and the blood delivers oxygen to the tissues throughout the body. But as blood volume falls, the oxygen delivery rate from lung to blood is markedly reduced and the tissues suffer from an oxygen debt. In the absence of oxygen, the tissues cannot utilize the oxygen-requiring Kreb's cycle metabolic reactions and instead must rely on anaerobic pathways to produce energy. The predominant anaerobic pathway culminates in the production of lactate from pyruvate. For this reason, in cases of reduced blood volume from hemorrhage, the level of lactate in the blood rises. The blood lactate also rises in the situation of dehydration (intravascular volume depletion). It also rises in the case of septic shock (due to infection) wherein blood vessels vasodilate. In this latter situation, the blood volume is not actually low, but due to the vasodilation, the “effective blood volume” declines markedly and blood lactate rises. Thus, a lactic acid sensor would be useful in all these situations: hemorrhage, dehydration and reduced effective blood volume from disorders such as septic shock.

Detecting OH by finding elevated blood lactate (lactic acid) concentrations could allow for the institution of rapid resuscitation (administration of fluids and blood, etc.) that may reduce the mortality rate. When a medic or emergency medical technician (EMT) is called to provide care for an injured person, one of the first procedures that he/she carries out is to insert a catheter in a vein, often in the arm. Thus, an in situ sensing element coupled to a catheter may provide a useful arrangement in such an environment, allowing for early detection of a potentially life-threatening event.

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIG. 1 illustrates a sensing device in accordance with an embodiment of the present invention in which each panel shows different layers of the device;

FIG. 2 illustrates a sensing and drug delivery device having multiple sensing zones in accordance with an embodiment of the present invention;

FIG. 3 illustrates a sensing and drug delivery device having multiple sensing zones in accordance with an embodiment of the present invention;

FIG. 4 illustrates a sensing and drug delivery device in accordance with an embodiment of the present invention;

FIG. 5 illustrates a sensing device coupled to a sensor module in accordance with an embodiment of the present invention;

FIG. 6 illustrates a winged holder for a sensing and drug delivery device in accordance with an embodiment of the present invention;

FIG. 7 illustrates a flat sensing device having multiple sensing zones in accordance with an embodiment of the present invention;

FIG. 8 illustrates a sensing and drug delivery device in accordance with an embodiment of the present invention;

FIG. 9 illustrates a sensing and drug delivery device in accordance with an embodiment of the present invention in which, in Panel A, the sensing and drug delivery functions are integrated into a single tube, and in which, in Panel B, the sensing and drug delivery functions are separated into different tubes; and

FIG. 10 illustrates a device in accordance with an embodiment of the present invention inserted subcutaneously.

### DETAILED DESCRIPTION OF EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the present invention, the phrase “A/B” means A or B. For the purposes of the present invention, the phrase “A and/or B” means “(A), (B), or (A and B)”. For the purposes of the present invention, the phrase “at least one of A, B, and C” means “(A), (B), (C), (A and B), (A and C), (B and C), or (A, B and C)”. For the purposes of the present invention, the phrase “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

Embodiments of the present invention may be provided with features described herein individually, or in any suitable combination, whether or not specifically described in combination, based on the teachings herein.

Embodiments of the present invention provide for analyte sensing combined with drug delivery in an integrated system. In an embodiment, a device may be utilized to sense an analyte, and in response to a measurement obtained therefrom, introduce a controlled amount of a drug to a user as a corrective action.

An embodiment of the present invention teaches a closed loop system in which a sensor and a drug delivery device are integrated into a single hollow structure. An alternative embodiment consists of two or more elongated structures (for example, a sensor and a drug delivery device) that are in close proximity and are each connected to one or more parts placed against the skin of the user.

For the purposes of the present invention, the term “drug” should be construed broadly to refer to any substance or infusate presented for treating, curing or preventing a disease or condition in animals, such as mammals, for example humans. In an embodiment, a drug may be used for restoring, correcting, and/or modifying physiological functions. Thus, examples of drugs in embodiments of the present invention include insulin, blood, saline, water, etc., as well as various pharmaceuticals, nutraceuticals, etc.

In an embodiment of this invention, the sensing portion of a device and the drug delivery portion of the device may be integrated into one hollow structure. In an embodiment, a drug (for example, insulin) may be delivered into a mammalian body through the distal lumen of the device. In an embodiment, an analyte (for example, glucose or lactate) whose serial concentrations are given to a controller in order to determine the drug delivery rate, may be measured at a site proximal to where the drug is delivered. The orientations of the various sites being proximal or distal are for exemplary purposes, and may be modified as desired in accordance with the teachings of embodiments of the present invention.

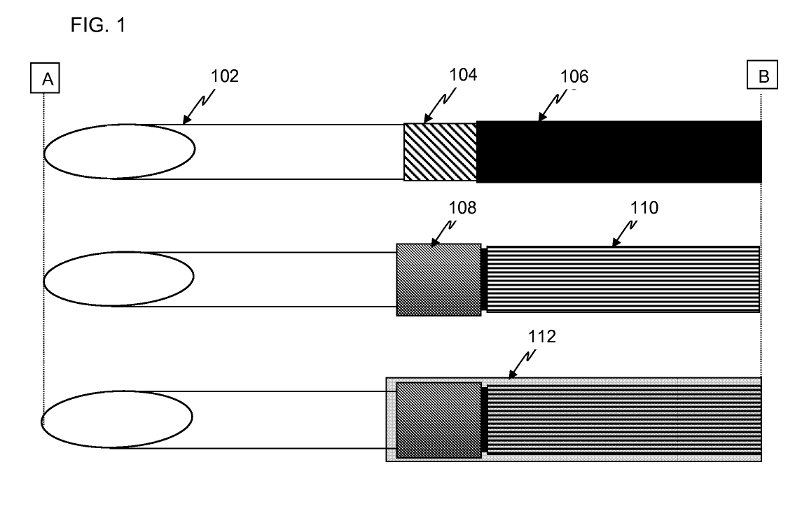

A basic design of an embodiment of the present invention is shown in FIG. 1. In the embodiment of FIG. 1, there are multiple layers and for this reason, the figures are divided up into three panels, with only the bottom panel having all the layers. Shown in the upper panel of FIG. 1 is a hollow structure 102 that extends from point A to point B. In an embodiment, structure 102 is a tube made from a non-conducting polymer, but it may also be made from a conducting metal, a conducting polymer, glass, or other suitable materials. In an embodiment, suitable polymers for forming a tube include fluoropolymers, polyethylene, or polymers used for intravenous catheters.

For the purposes of the present invention, the term “hollow” when referring to various structures according to embodiments of the present invention encompasses a broad range of cross-sectional sizes and shapes. In general, a hollow structure is one that has one or more passages through which fluid or gas may flow, regardless of whether the passages are straight, curved, bent, irregular, etc.

Material 104 may be present on all or part of the outer surface of structure 102 and, in an embodiment, this material may be platinum, but may also be gold, silver, palladium, tantalum or carbon. In an embodiment in which material 104 is carbon, it may be glassy carbon, carbon fibers, graphite or carbon nanotubes. In an embodiment, material 104 extends proximally to point B. In an embodiment, material 104 serves as the indicating electrode of the sensor and may be applied to structure 102 by electroplating, electroless plating, sputtering, metal evaporation, plasma vapor deposition, photolithography, or pad printing of metalized ink, such as platinum ink dispersed in a polymer matrix, or by other methods known to persons skilled in the art.

In an embodiment of the present invention, an indicating electrode may have a variety of shapes and sizes. An indicating electrode may encircle a central tube in one or more rings, or may be disposed on the tube without encircling the tube, or there may be a combination of arrangements. In an embodiment of the present invention, an indicating electrode may form a trace that extends along a tube or flattened surface or substrate.

In an embodiment of the present invention, an insulating layer (dielectric) (enumerated here as structure 106) may exist over part of the surface of material 104. Dielectric 106 may be placed over material 104 and/or on structure 102 by one of several methods, including but not limited to dip coating, spray coating, ink jet printing, or photolithography. In an embodiment, dielectric 106 may be crosslinked by ultraviolet or heat curing to make it more robust and less susceptible to dissolution by solvents or environmental extremes.

More superficial layers of the device are shown in the middle panel of FIG. 1. Layer 108 is a surface that serves as the reference electrode of the analyte sensor and, in an embodiment, may be made from silver. The reference electrode may be applied by electroplating, electroless plating, sputtering, metal evaporation, or by other methods known to persons skilled in the art. In an embodiment, a silver reference electrode may have a layer of silver chloride formed on the surface which may be carried out by the use of, for example, ferric chloride treatment or electrolysis. In the latter method, a current is passed through the silver during immersion in a solution of HCl and KCl, and is properly termed electrolytic chloridization.

In an embodiment of the present invention, a silver/silver chloride layer may also be applied to the all or part of the surface of a module that contacts the skin. In such an embodiment, the reference electrode may contact the skin in a fashion similar to common electrocardiographic electrodes.

In an embodiment, reference electrode 108 may be applied concentrically around part or all of dielectric 106 and/or part of material 104. In an alternative embodiment, the indicating electrode and the reference electrode may be applied as flattened wires that are not concentric to one another. In such an embodiment, the indicating electrode and the reference electrode may be co-extruded with the basic substrate.

In an embodiment, a reference electrode may be silver, silver/silver chloride, stainless steel, or other suitable materials in accordance with the teachings of the present invention. In an embodiment, a reference electrode may be a solid metal or may be deposited in the form of an ink. In an embodiment, a reference electrode may have an exposed area greater than an exposed area of an indicating electrode, for example, at least 3, 4, or 5 times as great an exposed area.

In an embodiment, an additional electrode, such as a counter electrode, may be utilized. In an embodiment in which a counter electrode is utilized, current may flow through the counter electrode rather than through the reference electrode thus decreasing the potential for alteration of the polarizing voltage.

In an embodiment, a series of membranes may be applied over material 104 and, collectively, these membranes may be termed the transduction layer 110. The basic nature of these layers in an embodiment of the present invention may be found in two issued patents, U.S. Pat. No. 5,165,407 (Implantable Glucose Sensor, Wilson et al.) and U.S. Pat. No. 6,613,379 (implantable Analyte Sensor, Ward et al.), the contents of which are hereby incorporated by reference. In an embodiment, these layers may include, as the innermost layer, a specificity membrane that allows hydrogen peroxide to permeate through to the underlying electrode but does not allow interfering species such as ascorbate, acetaminophen and uric acid to permeate. This specificity membrane may be made from sulfonated polyethersulfone, as taught in U.S. Pat. No. 6,613,379, or from other compounds, such as cellulose acetate or NAFION, etc. In an embodiment, superficial to the specificity membrane may be a catalytic membrane that enzymatically catalyzes the formation of hydrogen peroxide. In one embodiment (in which the analyte is glucose), this catalytic membrane may contain glucose oxidase that has been immobilized with the crosslinking agent glutaraldehyde in the presence of a protein extender such as albumin. If lactic acid is the analyte, the enzyme may be, for example, lactate oxidase or lactate dehydrogenase. Construction of certain enzyme-based sensors is well known in the art and many such enzymes that may be used for analytical purposes for various analytes are known and contemplated within the scope of embodiments of the present invention.

In an embodiment, permselective membrane 112 may be the most superficial layer and may cover reference electrode 108 in addition to an underlying catalytic membrane. A permselective membrane serves the role of regulating the permeation of the analyte of interest and of oxygen. For example, if glucose is being measured, in an embodiment of the present invention, a permselective membrane may be highly permeable to oxygen but minimally permeable to glucose. In this manner, stoichiometry is maintained and the potential of becoming oxygen limited at high glucose concentrations may be minimized. In an embodiment, membrane 112 may be made of a polyurethane that has hydrophilic blocks through which glucose permeates and hydrophobic blocks through which oxygen passes. In an embodiment, a permselective membrane may have a silicone or fluoropolymer moiety to assist with oxygen permeation. In an embodiment of the present invention, a permselective membrane may possess a hydrophilic moiety, such as a polyethylene oxide or polyethylene glycol to assist with analyte permeation. Many other such permselective membranes have been described and are known to persons skilled in the art and contemplated within the scope of embodiments of the present invention. For example, PCT Publication No. WO2004/104070 and U.S. patent application Ser. No. 11/404,528, entitled “Biosensor Membrane Material,” filed on Apr. 14, 2006, provide details pertaining to particular components of suitable permselective membranes, the entire disclosures of which are hereby incorporated by reference.

In an embodiment in which structure 102 is a metalized surface, the entire surface may be covered with a specificity membrane in order to avoid interference from oxidizable compounds that may generate a current when a polarizing bias is applied.

In an embodiment of the present invention, a sensing and/or drug delivery tube may be, for example, 1-2 inches in length or longer, such as a hollow wire or tube, peripherally inserted central catheter, jugular or subclavian central catheter, Swan-Ganz, or other catheter, etc. In an embodiment, a tube may have a variety of cross sections, both in size and shape, depending on the particular desired application.

An alternative method of fabricating a device in accordance with an embodiment of the present invention, rather than beginning with a hollow structure, is to begin with planar structures. For example, base substrate 102 may be a planar structure. In such an embodiment, the individual layers may be applied to substrate 102, then as a final step, the planar structure may be wrapped into a hollow structure, for example, around a mandrel. In such an embodiment, a seam may be created as the two edges are joined. The process of photolithography (using negative or positive photoresists) is particularly well-suited for adding chemical layers to planar structures although other methods may be utilized according to the teachings herein.

Yet another method of fabricating a device in accordance with an embodiment of the present invention is the joining together of more than one hollow structure. For example, substrate 102 on which a metal surface may be applied may be the first tube. A second tube could be a shorter tube on which a silver/silver chloride reference electrode and multiple transduction membranes were deposited. During fabrication, the second tube may be applied directly over the first tube in a nested, telescoping arrangement.

In an embodiment, an alternative to having a single lumen is to have more than one lumen. In such an embodiment, one lumen may be used to serve as a conduit through which a reference electrode (for example, silver/silver chloride) may enter the tissue. The use of multiple lumens also provides the advantage of allowing more than one drug or different mixtures or concentrations of drugs, etc. to be infused.

In an embodiment of the present invention, an alternative to having one indicating electrode (e.g. a platinum surface) on which sensing compounds may be applied is to have multiple indicating electrodes, each of which has sensing compounds applied. In such a configuration, more than one analyte may be measured concurrently.

In an embodiment, multiple indicating electrodes may be created by adding sequential layers of insulating dielectric material to more proximal portions of the sensor and upon each dielectric layer, adding an additional indicating electrode. In this embodiment, each of the nested, telescoping indicating electrodes may be covered with an enzyme that allows it to measure a specific analyte. In addition to the enzyme, in an embodiment, each indicating electrode may also be covered with a specificity membrane directly adjacent to the electrode surface and a permselective barrier membrane superficial to the catalytic enzyme layer. In an embodiment, one reference electrode may service all the indicating electrodes.

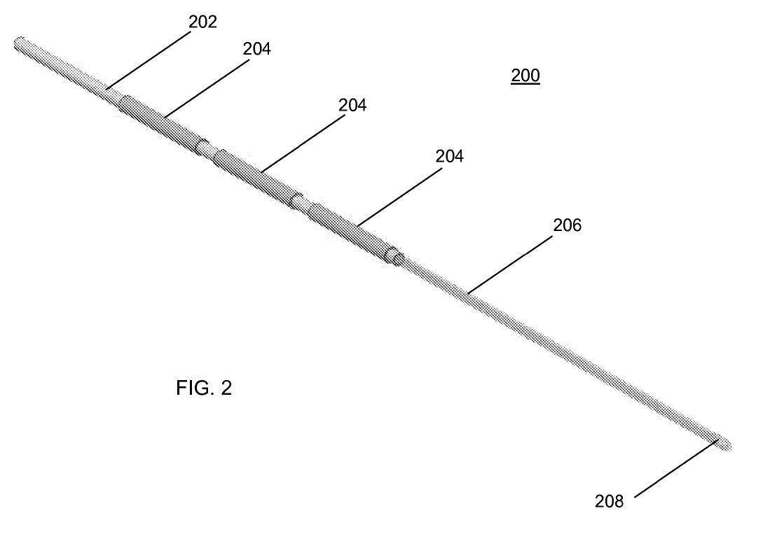

An embodiment of the present invention is shown in FIG. 2. FIG. 2 shows a sensing device 200 with three exemplary sensing zones 204. Sensing device 200 has a core 206, for example constructed of a flexible tube, with an outer layer 202, of, for example, platinum. At one end of sensing device 200 is found a port 208, for example, for delivering a drug when in use.

In an embodiment of the present invention, sensing zones 204 may be used to sense one or more analytes. In an embodiment, for each analyte to be sensed, a sensing zone 204 may have an analyte responsive enzyme and an indicating electrode to provide an indication of the concentration of analyte being measured.

In an embodiment, a tube, such as shown by tube 206, may be constructed from a metal, polymer, glass, etc. In an embodiment, a tube may be flexible, meaning that it may undergo repeated flexure without breaking, making it usable for an extended period of time within a body, such as days or weeks.

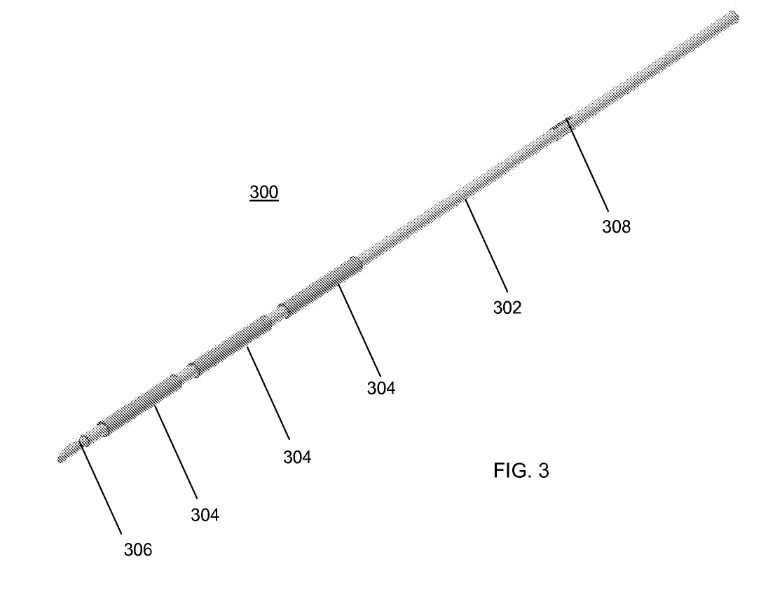

An embodiment of the present invention is shown in FIG. 3. FIG. 3 shows a sensing device 300 with three exemplary sensing zones 304. Sensing device 300 has a layer 302, of, for example, platinum. Along sensing device 300 is found a port 308, for example, for delivering a drug when in use. In an embodiment, a plug 306 is also provided, which may be removable, or rather the device may be configured such that the device is closed or fused at one end.

In an embodiment of the present invention, any suitable number of sensing regions may be provided, such as 1, 2, 3, 4, or more. In an embodiment, more than one port may be provided, for example, each connected to a different lumen thus enabling the introduction of more than one drug through a dedicated, or at least differentiated, lumen. In an embodiment of the present invention, a lumen may be differentiated by branching, and/or by being divided into more than one passage by one or more dividing wall or membrane.

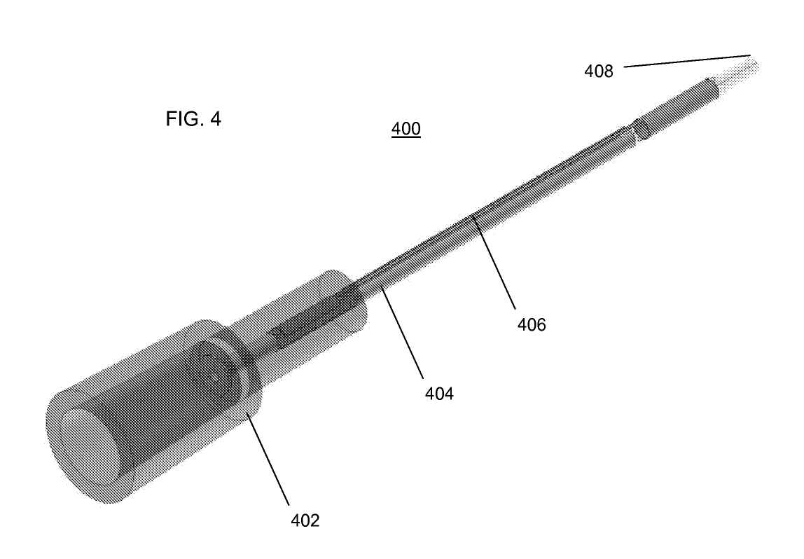

FIG. 4 shows an embodiment of the present invention in which a sensing device 400 is shown with an attachment mechanism 402, such as a luer lock, and various traces 404 and 406. Traces 404 and 406 are shown not fully concentric to each other, or to the underlying tube, but, in embodiments may be concentric to each other. For the purposes of the present invention, the term “trace” is to be construed broadly to refer to any electrically conductive path, and may be in a variety of physical arrangements. At one end of sensing device 400 is found a port 408, for example, for delivering a drug when in use. A sensing membrane (not shown) having one or more layers may further be applied to the outside of the traces according to an embodiment of the present invention.

In an embodiment of the present invention, multiple wires may be imbedded in the jacket wall of a tube, for example, by way of dual extrusion. In an embodiment, either the same materials may be used or materials of differing temperature and mechanical properties may be used, that is, the first extrusion may be, for example, of poly tetrafluoroethylene, then wires either round or flat may be fed in and laid on the tetrafluoroethylene and then a second extrusion applied in-line, immediately behind the first extruder head of polyurethane or some other lower temperature material that will not re-flow or melt the first extrudate.

In an embodiment, imbedded wires may be accessed by laser or exposed by another method, such as another sort of energy beam or mechanical abrasion, and used as a biosensor(s). In an embodiment, the wires may be used as the connector wires between an otherwise broad-band sensor site applied to the surface at the distal tip and the connection points required for termination at the proximal end.

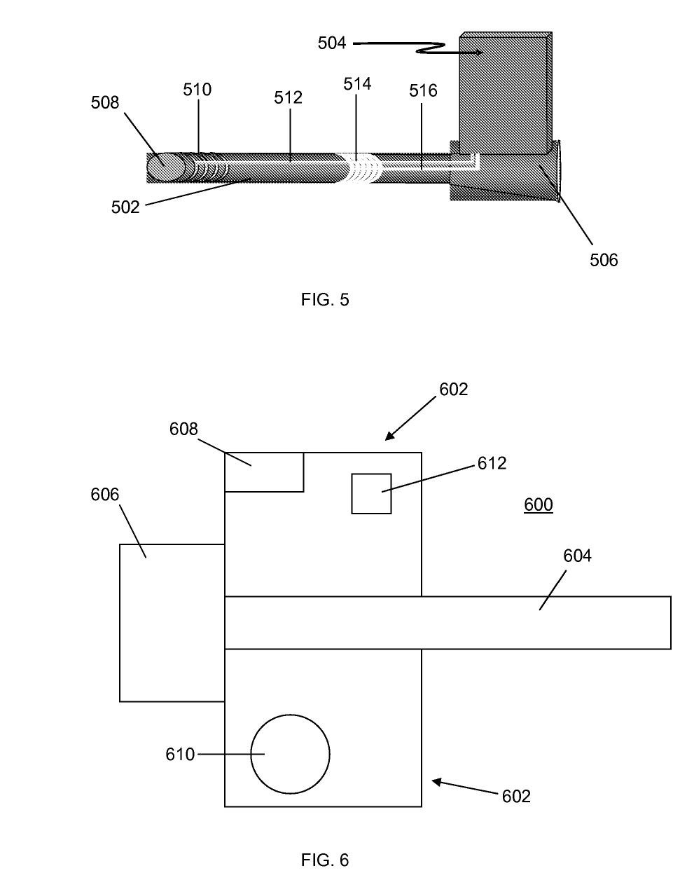

FIG. 5 shows an embodiment of the present invention, with a tube 502, such as a catheter, connected to a sensor module 504. Tube 502 has a hub 506, to which sensor module 504 is attached, and a distal drug delivery port 508. On the outside of tube 502 may be found an indicating electrode 510 electrically connected to sensor module 504 via trace 512. On the outside of tube 502 may also be found a reference electrode 514 electrically connected to sensor module 504 via trace 516. Although electrodes 510 and 514 are shown as multiple rings, various numbers of rings, and/or various arrangements of electrodes, are contemplated within the scope of embodiments of the present invention.

FIG. 6 shows a device 600 having a winged holder 602 for maintaining a tube 604, such as a catheter, in contact with the skin of a user. Winged holder 602 may be in a variety of shapes and may, in an embodiment, be in the form of a bandage or a flex circuit. In an embodiment, holder 602 may have an adhesive backing to aid in securing the device to the skin of a user. Holder 602 may also have integrated circuitry such as antenna 608, battery 610, and transmitter 612. More or less circuitry may be provided in connection with holder 602 as desired for the particular application. In addition, device 600 has a module 606 in which additional circuitry may be housed, such as processing and analysis systems, in addition to drug delivery mechanisms, such as a pump, drug reservoir, etc.

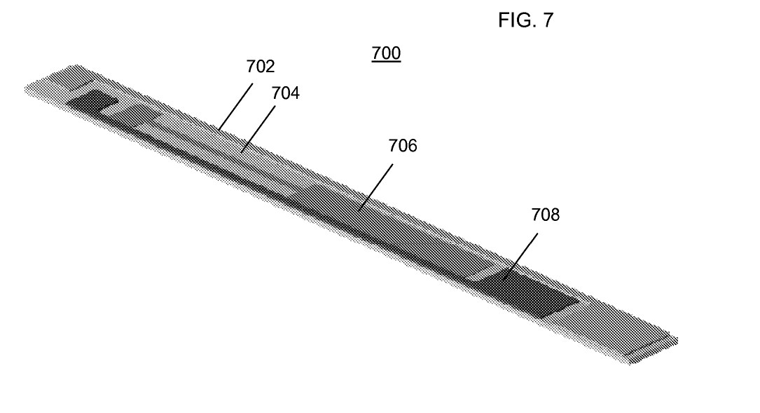

FIG. 7 shows a relatively flat sensing device 700 in accordance with an embodiment of the present invention. Device 700 has sensing zones 702 and 708 which may be configured in different shapes or arrangements, and may be connected in various ways to cathode 706. Zones 702 and 708, and cathode 706, are disposed on substrate 704, which may be composed of, for example, polyimide or KAPTON. Device 700 may be quite flexible and thus may be rolled around a mandrel or rolled into a tube itself, or other various shapes. Utilizing various sensing zones allows for sensing of one or more analytes as desired.

In an embodiment of the present invention, a substrate on which various sensing zones, electrodes and/or traces may be applied or formed may be in a variety of shapes and arrangements including flat, cylindrical, etc.

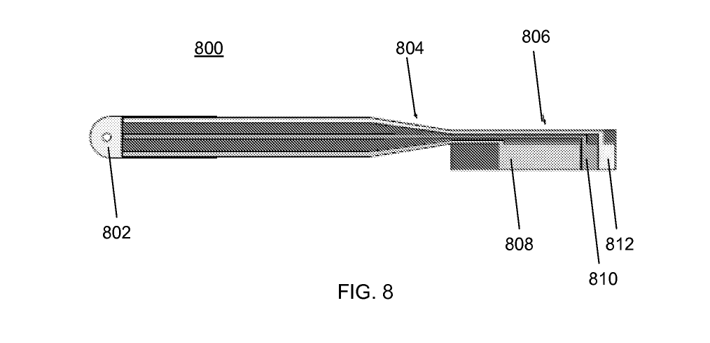

FIG. 8 shows sensing device 800 according to an embodiment of the present invention. Device 800 has sensing zones 810 and 812, which may be, for example, one or more noble metals working on conjunction with one or more analyte responsive enzyme layers. Utilizing various sensing zones allows for sensing of one or more analytes as desired. Device 800 also has cathode 808. In an embodiment, at region 806, the relatively flat features of the device allow the device to be rolled around a mandrel or rolled into a tube itself, or other various shapes (similar to as discussed above with respect to FIG. 7). In an embodiment, device 800, at region 804, may reside outside a body when in use, and may mate with an external drug delivery apparatus, for example, containing a reservoir, pump, etc. In an embodiment, device 800, at region 802, may be electrically connected to another device for power, analysis and/or display.

During operation of an automated endocrine pancreas according to an embodiment of the present invention, a positive polarizing bias may be placed on the indicating electrode(s) vs the reference electrode. In an embodiment, this bias may be between about 0.3 and 0.7 V. In an embodiment, the current that flows into the indicating electrode is obtained from oxidation of hydrogen peroxide at a noble metal surface and is proportional to the concentration of analyte (e.g. glucose) present in the tissue.

A device in accordance with an embodiment of the present invention may operate in several mammalian locations and types of tissue. For example, if placed in the subcutaneous tissue, it may measure glucose in the subcutaneous interstitial fluid and may deliver insulin into the subcutaneous tissue. It is important to understand that in embodiments of the present invention the sensing area may be separated from the drug delivery site. For example, if insulin is the drug that is delivered with this device, it may change the glucose concentration in the immediate vicinity. Insulin exerts its action in fat tissue (which is present in the subcutaneous location of mammals) by causing glucose to move from the interstitial fluid into the interior of fat cells (adipocytes). In addition, much of the insulin is absorbed into the bloodstream and thus leads to glucose uptake into cells throughout the body.

Because of its effect to draw interstitial glucose into cells, in the presence of high concentrations of local insulin, the interstitial glucose may fall to low levels. For this reason, if glucose is measured at a point very close to the insulin infusion site, the values obtained may not be representative of the whole body glucose concentration. Instead, the values obtained may be, to some extent, lower than that of the remainder of the body, since the concentration of insulin is typically highest at the local delivery site. For this reason, it may be beneficial for the sensing site to be separated from the drug delivery site. It is thought that in general, if insulin is infused into a specific site, that there is a zone of low glucose that surrounds that site. That zone has a radius of approximately 6-12 mm, but there are individual differences. Thus, in an embodiment, if the glucose is measured at least 6 mm, for example, at least 6-12 mm, such as at least 8-10 mm, away from the site of infusion, then the glucose concentration may be representative of the whole body peripheral adipose concentration. In the situation in which very high rates of insulin are being delivered, a larger separation distance may be beneficial, such as more than 12 mm, or more than 15 mm.

In an embodiment of the present invention, a combined sensing and drug delivery device may function when placed in a blood vein. In such a location, there is less of a need to separate the sensor from the insulin infusion port, since insulin does not exert its effect in the blood stream, but instead in the tissue after absorption from the blood stream.

In an embodiment, an intravenous insertion location of a device may be used for sensing lactate in the blood, which may serve as an indicator of hypoperfusion. Such a device may be used to introduce fluids and/or blood if a high level of lactate is measured. In an embodiment, lactate may be sensed near or away from one or more drug delivery ports.

In an embodiment of the present invention, when an individual is injured, such as in the situation of a military battle, motor vehicle accident, gunshot wound, etc., he or she is at risk of hemorrhage and death. In such a case, a lactate sensing catheter in accordance with an embodiment of the present invention may be inserted into a superficial vein. After insertion of a sensing catheter, a lactate sensor on the catheter may be calibrated. The attending health worker may obtain a drop of blood from the person (typically from the fingertip) using any widely available lancing device. In an embodiment, the drop of blood may be placed on a lactate sensing strip which is placed in a lactate measuring meter (e.g. Lactate Pro strip and meter). The resulting lactic acid level may be entered by the health worker into an electronic monitoring unit (EMU) to calibrate the lactate sensing catheter.

In an embodiment, the EMU then will display a continuous or nearly continuous lactate readout on its display, for example every minute. In an embodiment, the EMU may have alarm levels that may be set. For example, in an embodiment, one could set the EMU to activate an audible alarm when the lactate concentration exceeds a defined value, such as 2.5 mM. In an embodiment, when the lactate sensor (EMU) indicates rising lactate, the health worker may wish to obtain a confirmatory value with the fingerstick lactate meter.

In an embodiment of the present invention, when lactate concentration is found to be elevated, the health care team must act quickly because the patient may well have impending hemorrhagic shock. The patient may need to have blood or fluids administered and may need to have an abdominal exploration operation to rule out internal bleeding. A closed-loop system in accordance with an embodiment of the present invention facilitates rapid detection and correction of hypoperfusion as evidenced by elevated lactate levels in the blood.

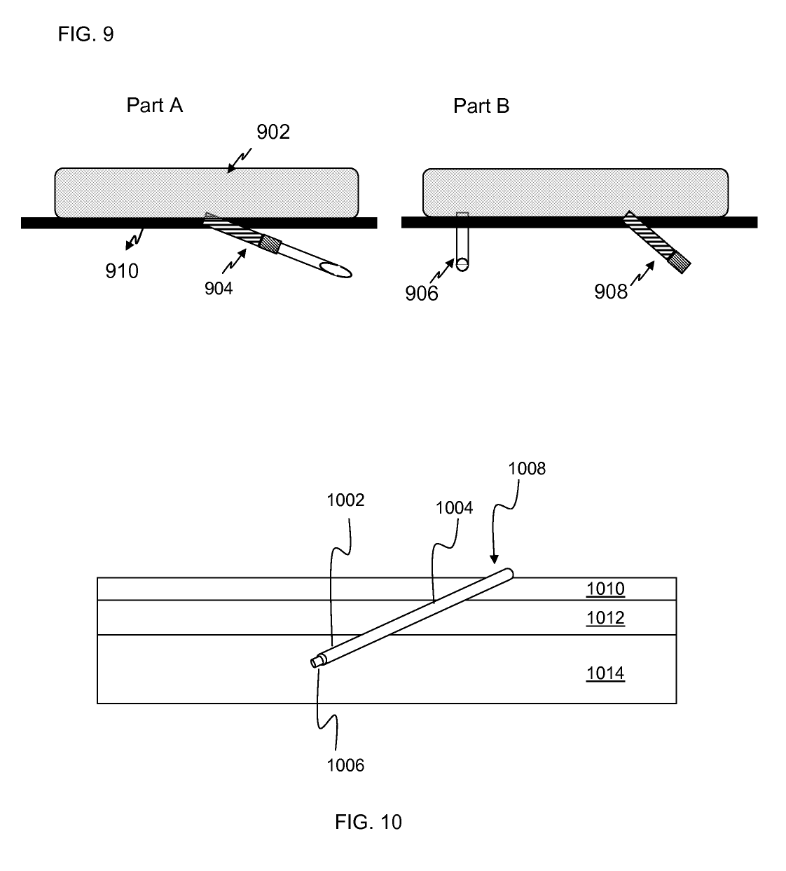

A method by which an embodiment of the device may be used may be understood by viewing the embodiments of FIG. 9. In Part A, a combined sensor/drug infusion catheter 904 has a diameter of about 75-300 microns, for example about 150-225 microns. Catheter 904 is attached to a device such as an on-skin electronic module 902. Module 902 rests on the surface of the skin 910 so that the tip of catheter 904 is located within subcutaneous fat. The distance between the skin surface and the depth of the device 904 is approximately 4-7 mm in an embodiment of the present invention. However, in embodiments of the present invention various angles of entry of a catheter with respect to the skin surface are contemplated, such as 90° or less, for example 10°, 20°, 30°, or 40°, which would impact the depth of penetration of the device with respect to the skin surface. In an embodiment of the present invention, in order to separate the sensing element from the insulin infusion port (and thus avoid the falsely lowered measurement of the analyte), the device may exit the module at an angle of, for example, 20-30°.

Another embodiment of the invention is shown in Part B of FIG. 9. In this embodiment, drug infusion catheter 906 is separated from the analyte sensor 908, and there are two sites from which the devices may exit from the on-skin electronic module. An advantage of this embodiment is that catheter 906 may be separated from the analyte sensing device 908 by a greater distance, thus lessening the risk of measuring an analyte concentration that is falsely low. In addition, each device may be shorter than the combined device shown in part A, since they are separated by their location within the module. The sensor 908 may either be hollow or solid.

In an embodiment, electronic module 902 has a component that provides a continuous polarizing bias to the metal electrode, for example of noble metal. In addition, the module may amplify the amperometric signal and may process the data in order to arrive at a calibrated analyte value. Alternatively, the signal may be transmitted to an external EMU where processing occurs. Either the module or the EMU may display and store the analyte data and may serve as the processor that deploys the algorithm by which the analyte data is used to determine a variable rate drug delivery rate.

In an embodiment of the present invention, there are several means by which devices may be inserted into the tissue. If inserted at a high rate of speed, there is no need for a separate trocar or needle to penetrate the skin. Alternatively, a stylet with a sharpened tip may be placed within the lumen of a hollow device. After penetrating the skin and subcutaneous tissue, the stylet may be withdrawn (to minimize pain and allow greater flexibility) or left in place. In the case of a solid device (sensor 908 for example may be solid), a hollow trocar may be placed around the sensor. After insertion into the tissue, the trocar may be withdrawn into module case 902 (as taught in U.S. Pat. No. 6,695,860 Transcutaneous Sensor Insertion Device, Ward et al., the entire contents of which are hereby incorporated by reference).

Alternatively, the trocar, if it contains a slot, for example a longitudinal slot, whether straight or spiral, may be completely withdrawn and removed from the module.

The drug that is delivered through the lumen may originate from a reservoir that may be located in one of several sites. For example, the drug reservoir may be part of module 902. In another embodiment, the drug reservoir is located at a more distant site and, in an embodiment, coupled to a pump, syringe, or other motive force for delivering a drug.

In a configuration in which a drug reservoir is located away from the module, the drug may originate from a commercially available insulin pump, such as those from the following companies: Medtronic, Smiths Medical, Animas, Sooil, or Nipro. In another embodiment, a glucose sensor may be combined with the Insulet OMNIPOD insulin delivery system in order to make a modified device that may both measure glucose and deliver insulin.

In an embodiment of the present invention, one or more drug delivery sites may be one or more ports located along the device, or at the end of the device, or both. In an embodiment in which a drug delivery port is provided along the device, a drug may be delivered into a body via the proximal part of the device and analyte sensing may take place beyond the drug delivery port at a more distal part of the device. In other embodiments, these orientations may be reversed.

In an embodiment of the present invention as shown in FIG. 10, a cross-sectional view of tissue with an inserted device is provided. The tissue is composed of epidermis 1010, dermis 1012, and subcutaneous tissue 1014. Device 1008 has a sensing region 1004, near or within which may also be a drug delivery port. Such a drug delivery port may be within, or may be proximal or distal to sensing region 1004. Alternatively, or in addition to that mentioned above, a drug delivery port 1002 may be provided. In an embodiment of the present invention, a plug 1006 may be provided to cap the end of device 1008. In an embodiment of the present invention, a removable plug may be provided, or alternatively, the device may be configured such that the hollow portion of the device extends only partially within the device thus effectively forming a cap or plug at one end of the device.

In an embodiment of the invention, a processor (that may be located in an electronics module such as structure 902) obtains analyte data (e.g. glucose data), computes an appropriate drug (e.g. insulin) delivery rate, and sends that information to a drug delivery pump, which then infuses the appropriate rate of drug through the hollow structure. In another embodiment, the processor communicates with the sensor and the drug delivery pump by telemetry, in which case it may be located distant from the apparatus that is worn on the body.

In an embodiment of the present invention, there is provided a device having a hollow structure configured for placement into the tissue of a mammal that has an outer surface on which are disposed compounds that are capable of responding to the concentration of an analyte by generating an electrical current; the compounds including a sensing compound, and the hollow structure containing a lumen through which a drug is capable of being delivered.

In embodiments of the present invention, a drug delivery rate may be based in part upon the concentration of an analyte.

In embodiments of the present invention, an analyte may be glucose and a drug may be insulin.

In embodiments of the present invention, an analyte may be lactate and a drug may be a circulatory volume expander such as crystalloid or colloid.

In embodiments of the present invention, an analyte may be lactate and a drug may be one that increases cardiac output.

In embodiments of the present invention, a sensing compound may be a redox enzyme.

In embodiments of the present invention, a hollow structure may be configured to be inserted subcutaneously, intravenously, or intraperitoneally.

In an embodiment of the present invention, there is provided a device having a structure configured to be placed on the skin of a mammal that is connected to at least one hollow drug delivery device and at least one analyte sensor, each of which is configured to penetrate skin, the exit point(s) of the drug delivery device and analyte sensor being separated from each other at a location which may be at the surface of the skin when in use, the sensor containing a redox enzyme.

In embodiments of the present invention, a drug delivery device and/or a sensor may be configured to terminate in subcutaneous fat.

In embodiments of the present invention, a drug delivery device and/or a sensor may each be configured to terminate in a blood vein.

In embodiments of the present invention, a distance of separation between exit point(s) of a drug delivery device and an analyte sensor may be about 6 mm or more.

In embodiments of the present invention, sensors may be capable of measuring one compound, or at least two different compounds.

In embodiments of the present invention, methods of inserting or attaching devices to a body to measure an analyte are provided with features discussed herein. In embodiments of the present invention, methods of making devices with features discussed herein are also provided.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A device, comprising:
a hollow structure configured for placement into the tissue of a mammal, said hollow structure having an outer surface, a proximal end and a distal end, and at least one lumen, said at least one lumen providing a passage through which a drug may be delivered to the mammal;
at least one indicating electrode disposed on at least a portion of the outer surface of said hollow structure; and
compounds disposed on at least a portion of said at least one indicating electrode, said compounds being responsive to a concentration of an analyte, said compounds including a sensing compound.

2. The device of claim 1, wherein said sensing compound comprises a redox enzyme.

3. The device of claim 1, wherein at least one of said at least one indicating electrode encircles said hollow structure in one or more rings.

4. The device of claim 1, wherein said at least one indicating electrode circumscribes and covers said hollow structure.

5. The device of claim 1, wherein at least one of said at least one indicating electrode comprises an electrical trace on said hollow structure.

6. The device of claim 1, wherein each of said at least one indicating electrode comprises at least one member selected from the group consisting of platinum, gold, silver, palladium, tantalum, and carbon.

7. The device of claim 1, wherein said hollow structure is coupled at said proximal end to a drug delivery apparatus.

8. The device of claim 7, wherein said drug delivery apparatus comprises a pump.

9. The device of claim 7, wherein said drug delivery apparatus comprises a drug reservoir.

10. The device of claim 1, where said at least one lumen comprises more than one lumen.

11. The device of claim 1, wherein said at least one lumen comprises a drug port at the distal end of said hollow structure.

12. The device of claim 1, wherein said at least one lumen comprises one or more drug ports along the device, proximal to the distal end of said hollow structure.

13. The device of claim 1, wherein the distal end of said hollow structure is closed.

14. The device of claim 1, wherein said hollow structure comprises a metal.

15. The device of claim 1, wherein said hollow structure comprises a polymer.

16. The device of claim 1, wherein said hollow structure comprises glass.

17. The device of claim 1, wherein said hollow structure is coupled at said proximal end to an on-skin electronics module comprising a transmitter.

18. The device of claim 17, wherein said on-skin electronics module further comprises a pump.

19. The device of claim 17, wherein said on-skin electronics module further comprises a drug reservoir.

20. The device of claim 17, wherein said hollow structure exits said on-skin electronics module at an angle of about 20-30° with respect to the lower surface of the on-skin electronics module which is configured to contact a user's skin.

21. The device of claim 17, wherein said on-skin electronics module further comprises a silver/silver-chloride layer on the lower surface of the on-skin electronics module, said layer configured to contact a user's skin.

22. The device of claim 1, wherein said compounds define one or more sensing regions, each sensing region being associated with an indicating electrode.

23. The device of claim 22, wherein each sensing region is associated with a different indicating electrode.

24. The device of claim 23, wherein each sensing region comprises a different sensing compound.

25. The device of claim 1, wherein said compounds comprise a series of membrane layers.

26. The device of claim 25, wherein said series of membrane layers comprises an innermost specificity membrane layer, an intermediate enzyme layer, and an outermost permselective membrane layer.

27. A device, comprising:
an on-skin electronics module configured to be placed on the skin of a mammal;
a hollow structure coupled to said on-skin structure and configured for placement into the tissue of the mammal, said hollow structure having a lumen, said lumen providing a passage through which a drug may be delivered to the mammal;
an analyte sensor coupled to said on-skin structure and configured for placement into the tissue of the mammal, said analyte sensor having compounds disposed on a surface thereof, said compounds being responsive to a concentration of an analyte by generating an electrical current, said compounds including a sensing compound; and
wherein said hollow structure exits said on-skin structure at a first exit point and said analyte sensor exits said on-skin structure at a second exit point, said first exit point being separated from said second exit point.

28. The device of claim 27, wherein said first exit point is separated from said second exit point by about 6 mm or more.

29. The device of claim 27, wherein said first exit point is separated from said second exit point by more than about 15 mm.
